(19) United States
(12) Patent Application Publication
(10) Pub. No.: US 2012/0130160 A1
(43) Pub. Date: May 24, 2012

(54) IMAGING SYSTEM WITH DISPOSABLE PART

(76) Inventors: Steen Borrye, Hillerod (DK); Merete Louise BORRYE, Hillerod (DK)

(21) Appl. No.: 13/133,694

(22) PCT Filed: Dec. 9, 2009
(86) PCT No.: PCT/EP2009/066725

§ 371 Date: Jan. 23, 2012

(30) Foreign Application Priority Data
Dec. 10, 2008 (DK) PA200801756

(51) Int. Cl. A61B 1/04

(52) U.S. Cl. 600/103

(57) ABSTRACT

An imaging system comprising a disposable part (1) comprising a camera device (5), said disposable part arranged to be suitable for allowing said camera device to be inserted into a body cavity, a reusable control module (8) in communication with said disposable part, said reusable control module comprising a display which displays image signals from the camera device in the disposable part, a timing device which generates time of use data of the disposable part, said time of use data representing the amount of time which the disposable part is in use, and said imaging system being arranged to prevent use of the disposable part if the time of use of the disposable part reaches a predefined level. The disposable part further comprises a non-volatile memory component which stores time of use data generated by said timing device and in that said reusable control module is arranged to continuously monitor the time of use data stored in the non-volatile memory component of the disposable part and to stop displaying image signals from the camera in the disposable part when the time of use data stored in the non-volatile memory component reaches said predefined level. In this way an imaging system is provided which is able to prevent using a disposable part which has been used on one patient on another patient.

The current invention relates to an imaging system comprising a disposable part with a camera device and a reusable control module comprising a display. The disposable part is arranged to be suitable for allowing the camera device to be inserted into a body cavity. The reusable control module is arranged to be in communication with the disposable part and is able to display the image signals from the camera device in the disposable part on the display. The system further comprises a timing device which generates time of use data of the disposable part, said time of use data representing the amount of time which the disposable part is in use. The imaging system is furthermore arranged to prevent use of the disposable part if the time of use of the disposable part reaches a predefined level. This ensures that the disposable part will not be reused for multiple patients. This also ensures that the lifetime of the disposable part is controlled. In this way, the device can be manufactured more cheaply since the demands on durability and long life can be reduced since the lifetime of the product can be predetermined.

It should be mentioned that in the current specification the term “reusable control module” is to be understood as an electronic unit with some control electronics for converting an image signal from the disposable part to an image signal which can be displayed on a display. In one embodiment, a display is built into the control module and in another embodiment, the control module is provided with an image output connection port which can be connected to a standard display. The wording of the claims “a re-usable control module comprising a display” is meant to cover both embodiments. In other words it could be interpreted as “a re-usable control module with a built in display or connected to a display.” The control module could furthermore be equipped with other user interface features, for example buttons, LEDS, speakers, etc.

It should also be noted that the term “timing device” should be understood as a device which keeps track of one or more “times” or “counts”. The timing device could therefore comprise many different setups. For example, the timing device could consist of one or more timers or counters or it could comprise a real time clock and a logic component to measure time differences.

The term “disposable part” should be understood as a part which is meant to be used on a single patient and then discarded.

It should also be noted that in this specification the phrase “time of use” should be understood as the total time in which the disposable part is actively in use. This should typically be defined as the time in which the image signal being transmitted by the disposable part is actually displayed on a monitor. For example, the time during which the disposable part is being connected to the control module or the time in which the disposable part is inactive between different steps of a medical procedure should not be included in the time of use. For example, if the disposable part is used for 5 minutes at the beginning of a 5 hour long surgical procedure and then again for 5 minutes at the end of the surgical procedure, the time of use as defined above would be 10 minutes.

### DESCRIPTION OF RELATED ART

An example of a basic imaging system as mentioned in the start of the introductory paragraph is a system comprising an endoscope. Endoscopes have been known for many years. A typical example of an endoscope is described in U.S. Pat. No. 4,846,153. In this type of endoscope a camera is mounted at the end of the endoscope. The camera transmits images to a control module with a display which shows the images to the user. Endoscopes are usually complicated electro-mechanical devices which are quite expensive. Due to their price, endoscopes are re-used multiple times by cleaning and then sterilizing them between uses. However, in recent years with the decreasing costs of electronic equipment and with the decreasing cost of manufacturing it has become possible to make imaging systems as mentioned in the opening paragraph with disposable parts comprising cameras. In the endoscope example, a re-usable monitor is connected to a disposable endoscope. Some examples of disposable parts with built in cameras are disclosed in EP 1804639, EP 1836949, EP 1928292, WO 2006/111730, WO 2007/081580 and WO 2007/089491.

One advantage of using disposable parts is that it is known in advance that the part will only be used on one patient. In this case the demands made to the longevity of the part are reduced. This allows the disposable part to be made from cheaper materials which do not have to be as durable. In addition, it is known that the device does not have to be sterilized and the device can therefore be made from materials which would otherwise not be able to withstand cleaning and sterilization procedures.

However, one of the main problems with using an imaging system with a disposable part is that there is a risk that an unknowing person could mistakenly sterilize a disposable part after it has been used and attempt to use it on a new patient. The danger in this case is that since the disposable part has not been designed to be used on more than one patient nor been designed to be sterilized, there is a risk that the disposable part will not function properly during the second use and cause a danger for the patient.

It is therefore desired to prevent multiple uses of the disposable part as best as possible. WO 2007/089491 as mentioned above provides the disposable part with a built in timer. The disposable part furthermore comprises electronics which monitor the timer. When the timer reaches a predefined value, the disposable part deactivates itself and becomes un-useable. EP 1928292 has a similar solution where the disposable part comprises a built in battery with a limited lifetime instead of a timer. When the battery expires the disposable part is rendered useless.

Endoscopes are used for many different applications. One of these applications is to help in establishing artificial respiration for patients. An endotracheal tube is placed over the flexible insertion portion of the endoscope and the flexible insertion portion is then inserted into the airway of a patient. The vision system in the tip of the endoscope allows the endoscope to be guided into place without danger for the patient. Once the endoscope and the endotracheal tube are in place, the endoscope can be withdrawn leaving the endotracheal tube in place in the airway of the patient. In the current specification, the embodiments disclosed are directed to this application, but it should be understood that the teachings of the current specification can apply to many other types of endoscopes as well.

### SUMMARY OF THE INVENTION

It is a first aspect of the current invention to provide an imaging system as mentioned in the introductory paragraph which is better than the prior art imaging systems. In particular it is an aspect of the current invention to provide an imaging system with a disposable part where the disposable part is disabled after a certain amount of use in a manner which gives a high degree of security to the system while still providing flexibility to the user of the imaging system.

This aspect is in part provided in that said disposable part further comprises a non-volatile memory component which stores time of use data generated by said timing device and in that said reusable control module is arranged to continuously monitor the time of use data stored in the non-volatile memory component of the disposable part and to stop displaying image signals from the camera in the disposable part when the time of use data stored in the non-volatile memory component reaches the predefined level. In this way, the imaging system can be made more flexible without increasing the costs of the system significantly.

In one embodiment, the reusable control module could be arranged to provide a visual or an audible warning to the user of the disposable part a predefined amount of time before the control module stops displaying image signals from the camera in the disposable part. In this way, it is prevented that the image signal suddenly disappears without any warning during a procedure.

In another embodiment, the control module could be arranged to provide information to the user of the disposable part, said information comprising the time of use data stored in the non-volatile memory component of the disposable part. The phrase “provide information to the user” should be understood in that the time information could be provided in many different ways. For example, it could be displayed on the display of the control module in a graphical or textual form. It could also be provided via an audible signal or as a special light signal. The person skilled in the art will understand that many different embodiments could be imagined.

In a preferred embodiment, the control module could be arranged to provide information to the user of the disposable part, said information representing the time remaining before the reusable control module stops displaying image signals from the camera in the disposable part. This will give a clear message to the user of the endoscope and prevent the undesired case where the image suddenly disappears. In one embodiment, the reusable control module continuously displays the time remaining in minutes and seconds or seconds.

In one embodiment, the timing device could furthermore generate information relating to the time at and/or the date on which the disposable part was first used, said information being stored in the non-volatile memory component of the disposable device. In this way, it is possible to take two different cases into consideration, both the total time of use of the device and the time elapsed since first use of the device. The time elapsed since first use of the device should be understood as being the time which has elapsed since the system first displayed an image signal from a particular disposable part. This could also be understood as a combination between the time of use data in addition to the standby time or storage time after the first use.

In one particular embodiment, the reusable control module could be arranged to continuously monitor the information stored in the non-volatile memory component of the disposable part relating to the time and/or date on which the disposable part was first used, and to stop displaying image signals from the camera in the disposable part when the time elapsed since the disposable part was first used reaches a certain predefined level.

In such an embodiment, the disposable part could comprise the timing device. This allows the timing to be independent of the control module used. The timing device could comprise two timers, a first timer which continuously updates the time of use data stored in the non-volatile memory component of the disposable part and a second timer which keeps track of the time elapsed since the disposable part was first used and stores the time elapsed since first use on the non-volatile memory component of the disposable device.

In an embodiment which comprises two timers in the disposable part and in which one of the two timers measures time elapsed since first use, it could be advantageous if the disposable part further comprises a power source which is used to provide power to the second timer. In one example, the power source could a super capacitor which is charged when the disposable part is connected to the control module.

In another particular embodiment, the reusable control module could comprise the timing device. In one such embodiment, the timing device could comprise a real time clock which generates time and/or date information and where the timing device could be arranged to write the time and/or date information of the first use of the disposable part to the non-volatile memory of the disposable part when the disposable part is first used and where the control module could continuously compare the current time and date information generated by the real time clock and the time and date information of the first use of the disposable part stored in the non-volatile memory of the disposable part in order to determine the time elapsed since the disposable part was first used.

In a device where the timing device is located in the control module, the timing device could comprises a timer which continuously updates the time of use data stored in the non-volatile memory component of the disposable part as long as the disposable part is in use.

In order to save costs, the non-volatile memory component could be arranged as a part of the camera electronics.

As it is difficult for the disposable part itself to know when it is in use, the disposable part could comprise an on/off switch. In this way, it is possible to turn off the disposable part and to thereby reduce the time of use recorded in the non-volatile memory component of the disposable part. The disposable part could in an other embodiment comprise a motion sensor or other form for automatic sensor whereby the device can recognize when it is in use and then turn itself off when it is not in use. This could be an accelerometer which measures the motion of the disposable part or it could be a touch sensor which senses when somebody is holding the disposable part.

It should be emphasized that the term “comprises/comprising/comprised of” when used in this specification is taken to specify the presence of stated features, integers, steps or components but does not preclude the presence or addition of one or more other features, integers, steps, components or groups thereof.

### BRIEF DESCRIPTION OF THE DRAWINGS

In the following, the invention will be described in greater detail with reference to the embodiments shown by the enclosed figures. It should be emphasized that the embodiments shown are used for example purposes only and should not be used to limit the scope of the invention.

FIG. 1 shows a schematic perspective view of a first imaging system according to the invention.

FIG. 2 shows a schematic perspective view of a second imaging system according to the invention.

### DETAILED DESCRIPTION OF THE EMBODIMENTS

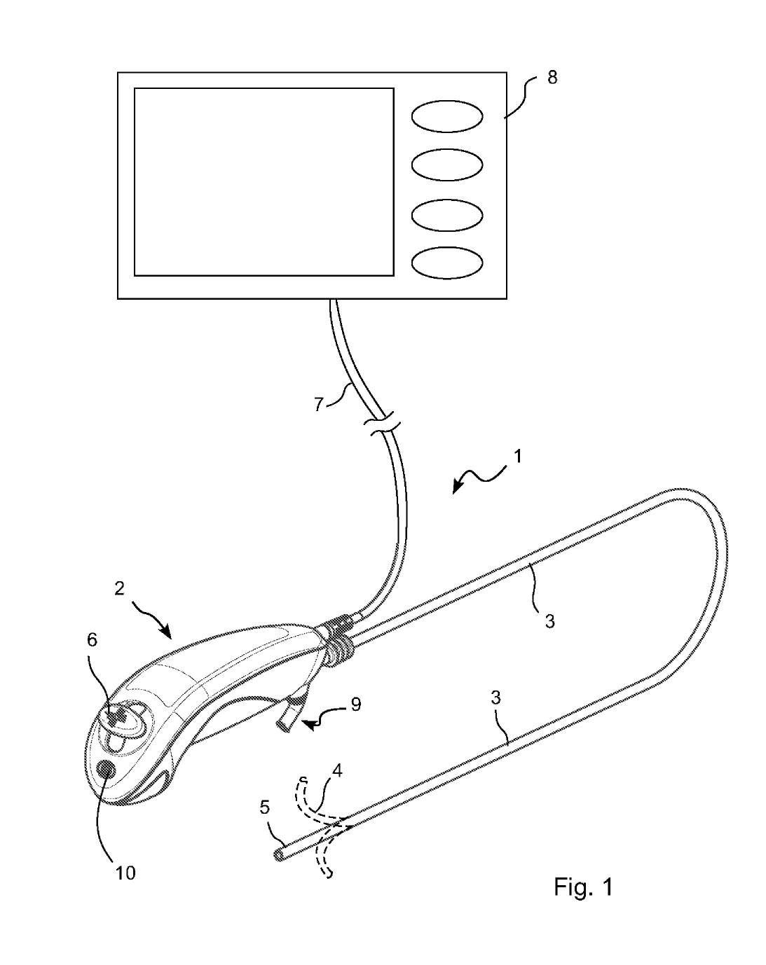

The endoscope 1 shown in FIG. 1 comprises a control handle 2, a flexible insertion portion 3, a bending portion 4 and a rigid tip portion 5. The control handle 2 comprises a joystick 6 which is connected to the bending portion via control wires (not shown) arranged within the flexible insertion portion and the bending portion. When the joystick is activated, the bending portion 4 bends as shown by the dashed lines in FIG. 1. The control handle also comprises an electrical cable 7 which is used to connect the endoscope to a reusable control module 8 comprising a power source and a video monitor/display. The control handle furthermore comprises an injection port 9 which is in communication with an opening (not shown) at the tip of the endoscope. The injection port can be used to inject fluids into the endoscope and out of the endoscope at the opening at the tip of the endoscope, for example a local anaesthetic or the like.

The flexible insertion portion 3 is arranged as a tube which is flexible and therefore bendable. However, the flexible insertion portion is stiff in the torsional and in the longitudinal direction. In this way, rotation of the control handle is transferred directly to the tip and allows the user of the endoscope to control the rotational position of the tip of the endoscope by twisting the control handle. A camera and a light source (not shown) are arranged in the rigid tip 5 (or camera housing) of the endoscope. The electrical signals from the camera and the power to the camera and the light source are transferred via wires arranged within the flexible insertion portion and the bending portion of the endoscope. An on/off switch 10 is placed on the control handle to be able to turn the endoscope on and off.

It should be noted that endoscopes comprising the above mentioned features are very well known to the person skilled in the art and further details will not be required by the person skilled in the art to understand and implement the current invention based on the following description and his or her common general knowledge.

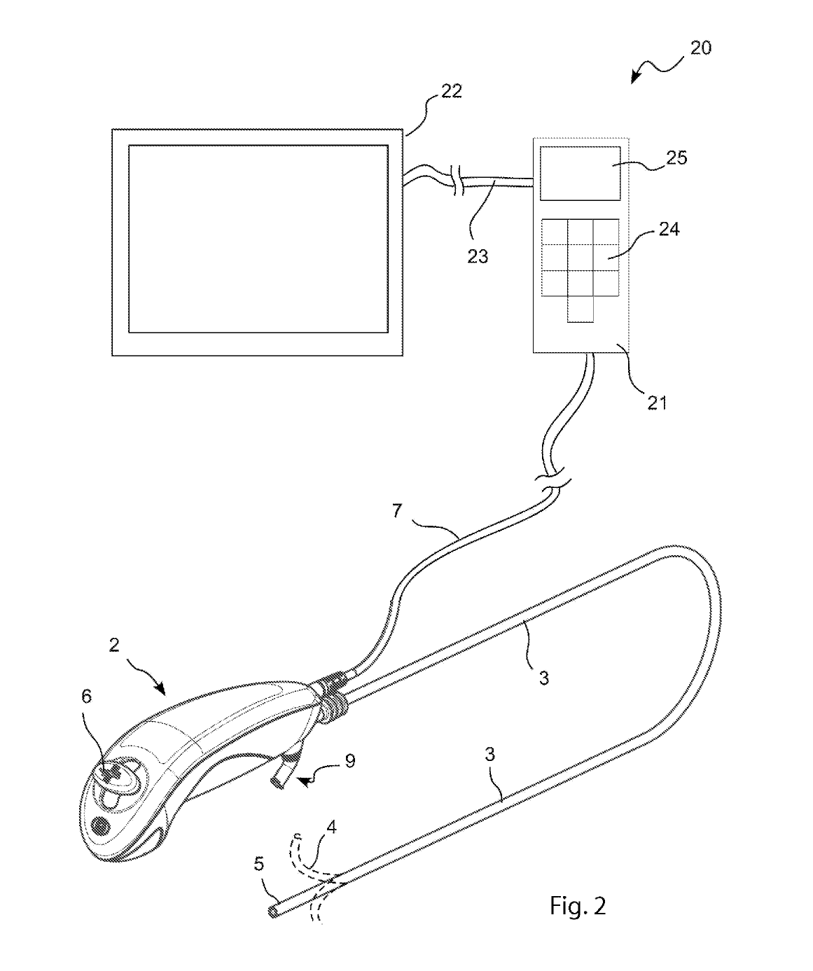

FIG. 2 shows a second imaging system 20 with a disposable part. The second imaging system is for the most part identical with the first imaging system shown in FIG. 1 with the exception of the re-usable control module 21. The same reference numerals have therefore been used for the same components. In this figure, the re-usable control module 21 does not comprise a built in display, rather the control module is only used for controlling the disposable part and converting the signal from the camera device in the disposable part into a standard video signal. A display 22 is then connected to the re-usable control module 21 via a signal wire 23. The display 22 could be one of many different types of standard displays. Such standard displays are available for many different purposes. The re-useable control module has a keypad 24 and a small text display 25 for displaying a simple menu structure and for displaying simple messages. It should however be obvious to the person skilled in the art that the form and features offered by the reusable control module can be quite different.

The display could also be a part of a more complex device, for example a stand alone computer. In this case, the communication between the control module and the computer could be via one or more different standard protocols, for example USB if cable based or BlueTooth® if wireless based. If the reusable control module is connected to a computer or other stand alone device, it would be possible for the control module to make use of the input means of the stand alone device. The reusable control module could also be a stand alone computer which interacts directly with the disposable part. For example, the disposable part could be connected to the re-useable part with a USB connection or a Bluetooth® connection. The person skilled in the art will understand that many different options are possible. In general however, it should be understood that the imaging system comprises a disposable part and a reusable part. The reusable part could be configured in many different ways as should be obvious from the discussion above and it could be connected to the disposable part in many different ways.

It should be noted that the embodiments described below will be implementable on many different types of physical components, for example those shown in either FIG. 1 or FIG. 2. The differences between the embodiments described below are related to the electronics within the physical components themselves. Specific details of the electronics are however not provided in this specification since the person skilled in the art of electronics will be able to implement the teachings of this invention based on the disclosure of this specification.

In a first embodiment, the disposable part comprises an electronic circuit.

Power to the electronic circuit is provided via the cable 7 from the control module 8. The electronic circuit could be the same electronic circuit which controls the camera device. The electronic circuit comprises a timing device which in this case is a counter (or timer) which counts the time which the disposable part is in use. The time which the disposable part is in use could be defined as the time in which the disposable part is actually transmitting an image signal to the control module or it could be defined as the time in which the control module is displaying an image from the disposable part. When the disposable part stops transmitting an image signal, or the control module stops displaying an image signal the counter will stop counting.

The counter value is stored on a non-volatile memory component in the electronic circuit. Since the memory component is non-volatile, the count value can be maintained even if the disposable device is disconnected from control module. In certain cases it could be imagined that the control module experiences a problem and needs to be exchanged during a procedure. In this case, the disposable part can be connected to a new control module and the procedure continued without loosing information as to the time of use.

In contrast to prior art embodiments where the disposable part monitors the count value and then deactivates itself when the count value reaches a predetermined level, in the current embodiment, the control module monitors the count value stored on the non-volatile memory component and stops displaying the image/video signal and/or cuts power to the disposable part when the count value reaches a predetermined value.

In one example, the disposable part is equipped with an on/off switch 10. The button in this embodiment is a toggle button which the user presses to start transmitting an image signal and then presses again to stop transmitting an image signal. In another embodiment, the control module could be equipped with a toggle button whereby the image signal from the disposable part could be controlled. By pressing the button, the image signal from the disposable part is displayed on the control module display. When the button is pressed again, the image signal from the disposable part is stopped being displayed on the control module. The on/off feature is useful for making the device more flexible. In certain cases, it could be imagined that halfway during an endoscope procedure, the patient experiences difficulties and the endoscope procedure needs to be stopped and another medical procedure started. In this case, it is useful to be able to stop the disposable part and then later on re-start the disposable part. This is not possible with the prior art type devices which are equipped with security means.

It should be noted that having the count value stored on a non-volatile memory component arranged in the disposable part allows the disposable part to be connected to various control modules while maintaining the count value. If the count value were stored on the control module, then it would not be possible to move the disposable unit from one control module to another.

It should also be noted that having the control module monitor the count value instead of having the disposable part monitor the count value gives many degrees of freedom to the designer of the imaging system. One example of such an advantage is in the case where the count value reaches the predetermined value before the medical procedure under way is completely finished. This is a serious problem with the prior art devices with security functions. It could very easily be imagined that halfway through a procedure the image signal suddenly stops due to the security system in the disposable part. In the current system, since the control module is the one controlling the signal, the software in the control module could be arranged to allow the security system to be overruled in certain cases. For example, the user of the system could be given a password which when typed into the terminal will re-establish the image signal. This will allow the user to finish the procedure. If the same functionality were to be programmed into the disposable part, the disposable part would become more expensive since it would require more input means such as a keyboard. Since the control module will be re-used multiple times, the costs of adding a keyboard to the control module will not be significant.

It should be noted that it might seem to be against the teaching of the invention to provide an override function on the control module. However, the main purpose of the security system is to prevent accidental re-use of the disposable part. While it would be possible to re-use a disposable part multiple times with a system having an override function as described above, it is maintained that the users of such systems would not make unwarranted multiple uses of a disposable part on purpose since they are aware of the risks involved.

Another benefit of having the control module monitor the count value is that the count value is always present in the control module as well. The count value can then, in one embodiment, be displayed on the display of the control module so that the user is always aware of how much time is left before the image signal is cut off. The count value can be displayed in many different ways, for example as a bar graph or as a digital value with hours/minutes/seconds remaining. In another embodiment, the count value is not displayed but an audible warning is provided a certain time before the image signal stops. In another example, a lamp or LED on the outer surface of the control module could be turned on a certain amount of time before the image signal stops. The person skilled in the art will be able to combine the above examples and find different combinations and ways of providing the count value to the user of the system.

In one concrete example, we will take the case of an imaging system where the disposable part is a disposable endoscope used to help place an endotracheal tube in the airway of a patient. In this example, the control module is programmed to stop displaying the image signal after 10 minutes of use. In this example procedure, the disposable part is connected to the control module before the patient enters the operating room. The disposable part is however turned off so that no image signal is displayed on the display of the control module. An endotracheal tube is placed over the flexible insertion portion of the endoscope. When the patient is ready, the endoscope is activated and the display of the control module starts displaying the image signal from the camera device in the endoscope. The endoscope can then be used to help place the endotracheal tube in the airway of the patient. Once the endotracheal tube is in place, the endoscope is removed and de-activated. During positioning and turning of the patient during the surgery, the anaesthesiologist may need to examine the airway of the patient again in order to make sure that the endotracheal tube has not been displaced. The endoscope is therefore re-activated and the endoscope used again. At the end of the procedure, the endoscope is disconnected from the control module and discarded.

In a second embodiment of an image system according to the current invention, the counter is not in the disposable part but is instead in the control module. A non-volatile memory component is however still present in the disposable part. The count value from the counter in the control module is constantly written to the non-volatile memory component in the disposable part. In this case, when the disposable part is disconnected from a control module and reconnected to another control module, the second control module can read the usage time from the disposable part and continue counting from the previous count stored in the disposable part. An advantage of this system over the previous embodiment is that the electronics on the disposable part can be made simpler. This will reduce the costs and complexity of the disposable part. This is especially true since in most cases, the camera device in the disposable part has control electronics which already comprise a non-volatile memory component. The software for controlling the camera device is usually stored on this memory component. The count value can therefore be stored on this already existing component without requiring additional memory components on the disposable part.

In a third embodiment, two counters are provided in the disposable part. A first counter is arranged as described in the first embodiment. The second counter is however arranged to keep track of the time which has expired from the very first time the disposable part was turned on. The second counter therefore also counts when the device is turned off or disconnected from the control module. The count value of the second counter is also written into the non-volatile memory component of the disposable part. The control module is arranged to check both count values in the non-volatile memory component of the disposable part. As in the first embodiment, when the first count value reaches a predetermined level, the control module stops displaying the image signal. However, in addition to monitoring the first count value, the control module also monitors the second count value. When the second count value exceeds a second predetermined level, then the control module also stops displaying the image signal. In other words, the control module constantly monitors the two count values in the non-volatile memory component and when either of the two values exceeds one of two different predetermined values, the control module stops displaying the signal.

In one example the first predetermined value is 10 minutes and the second predetermined value is 8 hours. The control module will stop displaying the image signal when either the first count value reaches 10 minutes or when the second count value reaches 8 hours.

The first count value is a count of the total time of use, ie the time in which the image signal from the camera device is being displayed on the control module. The second count value is the total time expired since the first use of the device. The two count values in combination provide a good security against misuse.

It should be noted that in order for the second counter to be able to measure time while the disposable device is not connected to a control module, the disposable device needs to have some sort of built in power source. One example of such a power source is a small battery. The battery will only be needed to power the counter so the power requirement is quite modest and a small cheap battery can be used. However, one of the problems with using a battery is that in many countries it is not allowed to dispose of batteries together with ordinary trash. Therefore, it is required to remove the battery from the disposable part before it is discarded. One way of getting around this problem is to use a super capacitor as a power source. The super capacitor can be charged up the first time the disposable part is connected to a control module. The size of the capacitor can be chosen to have a life time which is slightly higher than the second predetermined count value. In the example above, the super capacitor could for example have a life time which is at least slightly higher than 8 hours.

A slight variant of this third embodiment is to use a super capacitor as the second counter. The super capacitor would be arranged to be charged up the first time it is connected. However, the super capacitor would also be arranged not to be further charged after the first full charge. In this way, the control module could be arranged to monitor the power level on the super capacitor. When the voltage of the super capacitor drops below a certain value, then the control module could stop displaying the image signal.

Another slight variant of this third embodiment is to use super capacitors for both first and second counters. The super capacitor which represents the first count value would be arranged to be prevented from discharging when the device is switched off or disconnected from the control module. This could be arranged by connecting the first super capacitor to an on/off switch which is controllable by the user. The first super capacitor could also be connected to a switch which is on as long as there is power to the disposable part. The power to the disposable part could in this case be controlled by the control module.

A fourth embodiment is very similar to the second embodiment where the counter was built into the control module. In this fourth embodiment, the control module further comprises a real time clock. A time stamp is written into the non-volatile memory component of the disposable part the very first time the device is turned on. The control module can therefore constantly monitor the current time and compare it with the time stamp stored in the memory of the disposable part. When the time difference exceeds a certain value, for example 8 hours, the control module can stop displaying the image signal from the disposable part.

This embodiment has one drawback when compared with the third embodiment described above in that if the disposable part is transferred between two different control modules which have different clock states, ie the two control modules are not time synchronized, there will be a problem when comparing the value stored on the disposable part with the time on the new control module.

It is to be noted that the figures and the above description have shown the example embodiments in a simple and schematic manner. The internal electronic and mechanical details have not been shown since the person skilled in the art should be familiar with these details and they would just unnecessarily complicate this description. For example, no details have been provide as to how the control module communicates with the disposable part. However, it is maintained that the person skilled in the art of electronics will be able to provide this feature without any problems.

It is also to be noted that in the above detailed description, the word “counter” has been mainly used. However, the person skilled in the art should understand that this term should apply to any device which in some way keeps track of time. This could for example be a super capacitor which slowly discharges or a battery which slowly discharges. It could also be a timing unit which constantly measures the time difference between the current time and a time stamp. Or it could be a timer which measures the number of milli-seconds between start and stop.

In addition the above description has described the timer(s) or counters and the non-volatile memory component as being separate entities. However the person skilled in the art will know that these two components can be combined into a single component. For example, the counters could be provided with a built in non-volatile memory component which stores the counter value even when the counter looses power.

It should also be noted that in the above description, the disposable part has been described as comprising a camera device. Within this term, different types of devices could be imagined. For example CMOS video cameras, CCD video cameras, digital still picture cameras, analog cameras, etc could all be imagined. The person skilled in the art of vision systems will be able to provide further camera devices which are suitable.

1. An imaging system comprising
a. a disposable part (1) comprising a camera device (5), said disposable part arranged to be suitable for allowing said camera device to be inserted into a body cavity,
b. a reusable control module (8) in communication with said disposable part, said reusable control module comprising a display which displays image signals from the camera device in the disposable part,
c. a timing device which generates time of use data of the disposable part, said time of use data representing the amount of time which the disposable part is in use, and
d. said imaging system being arranged to prevent use of the disposable part if the time of use of the disposable part reaches a predefined level,
e. and said disposable part further comprises a non-volatile memory component which stores time of use data generated by said timing device, characterized
f. in that said reusable control module is arranged to continuously monitor the time of use data stored in the non-volatile memory component of the disposable part and to stop displaying image signals from the camera in the disposable part, and/or cuts power to the disposable part when the time of use data stored in the non-volatile memory component reaches said predefined level.

2. An imaging system according to claim 1, characterized in that said control module (8) is arranged to provide a visual or an audible warning to the user of the disposable part (1) a predefined amount of time before the control module stops displaying image signals from the camera device (5) in the disposable part.

3. An imaging system according to claim 1 or 2, characterized in that said control module (8) is arranged to provide information to the user of the disposable part (1), said information comprising the time of use data stored in the non-volatile memory component of the disposable part.

4. An imaging system according to any one of claims 1 to 3, characterized in that said control module (8) is arranged to provide information to the user of the disposable part (1), said information representing the time remaining before the control module stops displaying image signals from the camera in the disposable part.

5. An imaging system according to any one of claims 1-4, characterized in that said timing device furthermore generates information relating to the time at and/or the date on which the disposable part (1) was first used, said information being stored in the non-volatile memory component of the disposable device.

6. An imaging system according to claim 5, characterized in that said control module (8) is arranged
a. to continuously monitor the information stored in the non-volatile memory component of the disposable part (1) relating to the time and/or date on which the disposable part was first used, and
b. to stop displaying image signals from the camera in the disposable part when the time elapsed since the disposable part was first used reaches a certain predefined level.

7. An imaging system according to any one of claims 1 to 6, characterized in that said disposable part (1) comprises the timing device.

8. An imaging system according to claim 5 or 6 and claim 7, characterized in that said timing device comprises two timers, a first timer which continuously updates the time of use data stored in the non-volatile memory component of the disposable part (1) and, a second timer which keeps track of the time elapsed since the disposable part was first used and stores the time elapsed since first use on the non-volatile memory component of the disposable device.

9. An imaging system according to claim 8, characterized in that said disposable part (1) further comprises a power source which is used to provide power to the second timer.

10. An imaging system according to claim 9, characterized in that said power source is a super capacitor which is charged when the disposable part (1) is connected to the control module (2).

11. An imaging system according to any one of claims 1-6, characterized in that said control module (8) comprises the timing device.

12. An imaging system according to claims 5 and 11 or claims 6 and 11, characterized in that said timing device comprises a real time clock which generates time and/or date information and where the timing device is arranged to write the time and/or date information of the first use of the disposable part (1) to the non-volatile memory of the disposable part when the disposable part is first used and where the control module (8) continuously compares the current time and date information generated by the real time clock and the time and date information of the first use of the disposable part stored in the non-volatile memory of the disposable part in order to determine the time elapsed since the disposable part was first used.

13. An imaging system according to claim 11 or 12, characterized in that said timing device comprises a timer which continuously updates the time of use data stored in the non-volatile memory component of the disposable part (1) as long as the disposable part is in use.

14. An imagining system according to any one of claims 1-13, characterized in that said non-volatile memory component is a part of the camera electronics in the disposable part (1).

15. An imaging system according to any one of claims 1-14, characterized in that said disposable part (1) has an on/off switch (10).
